(19) United States
(12) Patent Application Publication
(10) Pub. No.: US 2004/0236210 A1
(43) Pub. Date: Nov. 25, 2004

(54) Image analysis method for vertebral compression curvature

(76) Inventors: Ming-Dar Tsai, Taipei (TW); Ming-Shium Hsieh, Taipei (TW)

Correspondence Address: RABIN & BERDO, P.C., Suite 500, 1101 14 Street, N.W., Washington, DC, 20005, US

(21) Appl. No.: 10/443,005

(22) Filed: May 22, 2003

(51) Int. Cl. A61B 5/05

(52) U.S. Cl. 600/427

(57) ABSTRACT

An image analysis method for vertebral compression curvature is disclosed for providing diagnosis analysis of the compression curvature. It makes use of the transverse sectional image with a concave feature of a vertebral body. After B-spline curves are approximated as ellipse-like surfaces, the method further evaluates the compression curvature of the canal. On the other hand, the center of the ellipse-like surface boundary obtained by approximation from different transverse sectional images of the vertebral body is used to reconstruct the centerline of the vertebral body by linear restoration. Such information is used to determine the curvature of the vertebral body. Moreover, the method can use the above-mentioned reconstructed vertebral body centerline to compare with other adjacent vertebral centerlines that have normal curvatures. In this manner, the method can help determine the type and extent of the spine under pressure or having a fracture.

### BACKGROUND OF THE INVENTION

FIELD OF INVENTION

The invention relates to an image analysis method and, in particular, to a method that diagnoses the spine compression curvature from the transverse sectional image of the spine.

RELATED ART

The diagnosis of spine compression curvature and particularly in determining the extent and type of the compression curvature has been the hardest part in medical sciences. However, the diagnosis information in this respect is the most valuable part in surgical operations and/or therapeutic procedures.

The diagnosis method for vertebral compression curvature, no matter from clinical findings or image diagnosis such as X-ray films, computed tomography (CT), and magnetic resonance imaging (MRI), cannot very accurately find out what the real problems are. The main reason is that the most accurate diagnosis method has to be companied with the three-dimensional image analysis for abnormal spines and the analysis between the problematic spinal sector and adjacent normal ones. Normal image diagnosis methods cannot provide desired accurate results.

In fact, the key information for vertebral compression curvature diagnosis is to be able to determine the extent and type of the vertebral compression. Generally speaking, the diagnosis result of the compression extent is to determine the anatomic curve deformation and canal compression extent. The diagnosis result of the compression type is to determine whether it is due to the abnormal pressure or breaking on the spine or pelvis or it is vertebral bending. An accurate diagnosis has to be able to do a good job on all the above things.

Therefore, how to use the mature computer software image analysis method to find the correlation between a problematic spinal sector and adjacent normal ones in order to determine the type and extent of the vertebral compression curvature is an important issue. This method can further help accurately performing surgical operations and subsequent therapeutic procedures.

### SUMMARY OF THE INVENTION

Since the B-spline curve has a good ability in approximating circles and arcs, the disclosed method can thus close the unclosed boundary extracted from the transverse sectional image of the spine for subsequent algorithmic analyses.

The method mainly uses the B-spline curve approximation to achieve the goal of computing the compression ratio and deformation level of the canal diameter in the transverse sectional image of the spine. On the other hand, the disclosed method can simultaneously extract different compression ratios obtained from several transverse sectional image of the same vertebral body, from which one can determine the compression curvature from the most serious compression state.

Of course, the disclosed method can compare the angles between the centerline reconstructed from different transverse sectional images and those of other adjacent normal vertebral bodies, in order to compute the necessary angles or displacements to make vertebral curvature corrections. Alternatively, comparing the lengths of the centerlines of the abnormal vertebral body and a normal one also enables one to determine the necessary height for restoration.

Consequently, the invention can solve the problems that normal clinical findings or usual image diagnoses in the past cannot provide accurate estimates for the extent and type of the spine compression curvature. With accurate diagnosis data, not only can a surgical operation become more accurate in positioning and operation procedures, the patient will also suffer less pain and side effects as a result of the accurateness of the operation. Moreover, analyzing the data can help reconstruct a three-dimensional image of the spine for subsequent medical references.

To achieve the above-mentioned objectives and effects, the disclosed method contains the following steps: extracting the transverse sectional images of a spine, computing and obtaining canal compression data from each transverse sectional image, finding the problematic spinal sector through the centerline analysis and computing the curvature of the problematic spinal sector, and evaluating the extent and type of the abnormal spine.

### BRIEF DESCRIPTION OF THE DRAWINGS

The invention will become more fully understood from the detailed description given hereinbelow illustration only, and thus are not limitative of the present invention, and wherein:

FIG. 1 is a flowchart of the main procedures in the disclosed method;

FIG. 2 is the flowchart for computing the canal compression according to the invention;

FIG. 3 is the flowchart for analyzing the spinal curvature according to the invention;

FIG. 4A to FIG. 4E are schematic views of computing the canal compression using the disclosed method;

FIG. 5A to FIG. 5E are schematic views of analyzing the spinal curvature using the disclosed method;

FIG. 6A to FIG. 6B are schematic views of transverse sectional images in an embodiment;

FIG. 7 shows the canal compression data of the transverse sectional images in an embodiment; and

FIG. 8 shows the curvature data of the transverse sectional images in an embodiment.

### DETAILED DESCRIPTION OF THE INVENTION

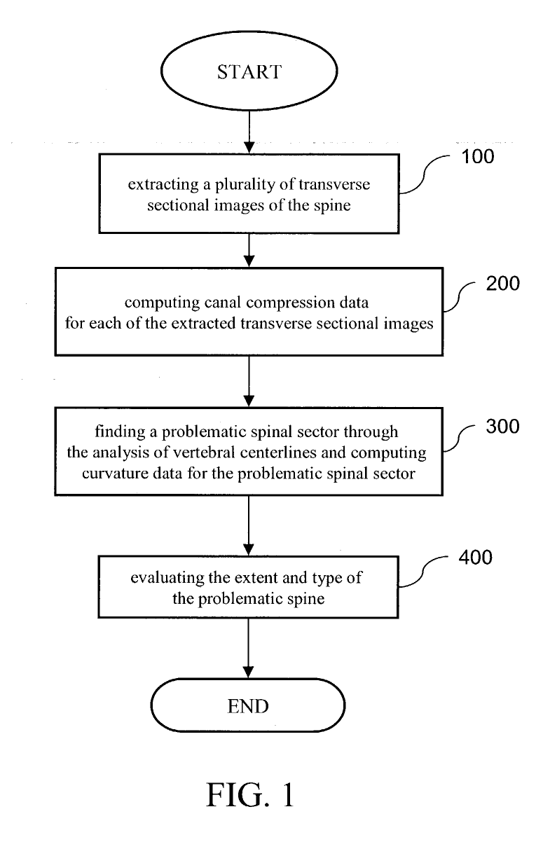

The invention discloses an image analysis method for vertebral compression curvature. It is primary used to perform diagnostic analysis of the vertebral compression curvature caused by pressure or fracture. First, we use FIG. 1 to explain the main procedure of the disclosed method.

In the beginning, we use computed tomography (CT) or magnetic resonance imaging (MRI) to extract transverse sectional images of the spine to be analyzed (step 100). In general, the extraction location, extraction spacing, and extraction amount are different as the results obtained from preliminary X-ray films vary. Each transverse sectional image is computed to obtain the compression data of the canal in it (step 200). Such data include the canal diameter, the three-dimensional coordinates of the canal center, and so on. This is because the canal diagonal part of the spine is most likely to be depressed by external forces and to be deformed. Therefore, the method uses this principle to compare the diameter variation among adjacent transverse sectional images to determine whether each vertebral body in each spinal sector is normal. Detailed information of this part will be further explained later with reference to FIG.

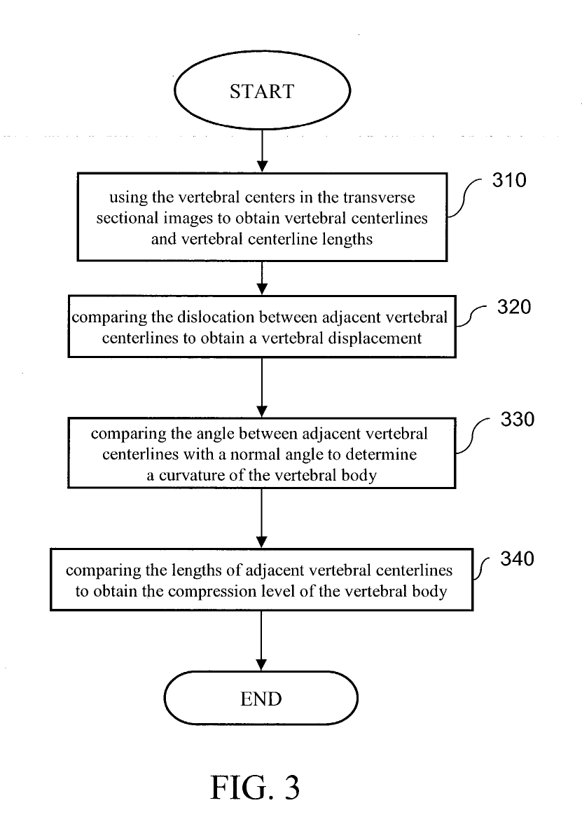

2. Through the analysis of the vertebral centerline, the method finds the problematic spinal sector and computes the curvature data of that sector (step 300). Such data include the average canal diameter, the spinal height, and the three-dimensional coordinates of the vertebral centerline. Since the angles and lengths of the vertebral centerlines in normal spinal sectors are roughly the same, the disclosed method can compare those of adjacent vertebral centerlines to estimate the compression curvature, which is to be explained in further detail later with reference to FIG. 3. Finally, the extracted data are used to evaluate the extent and type of the problematic spine (step 400).

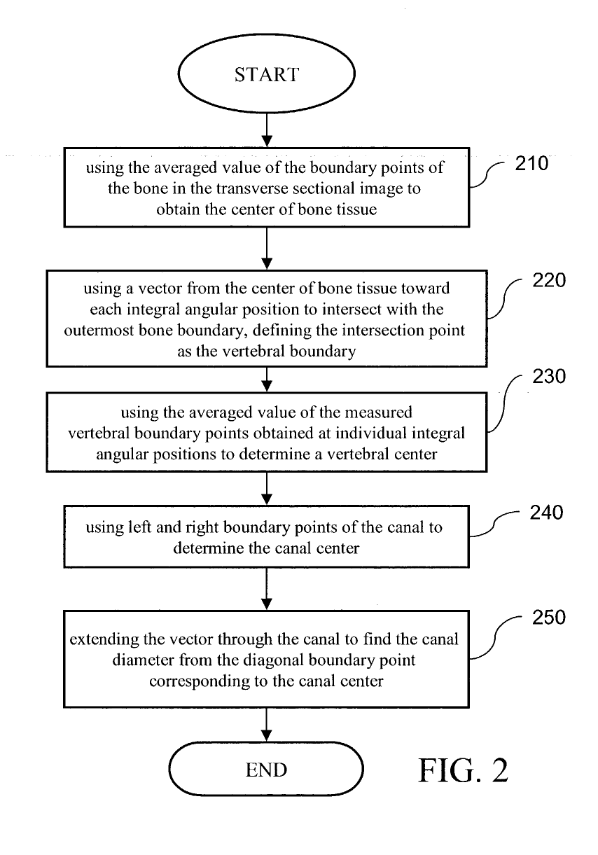

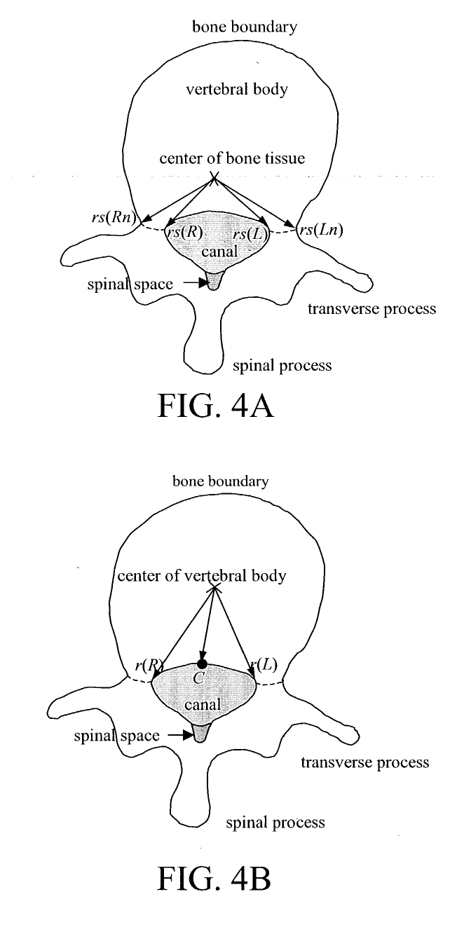

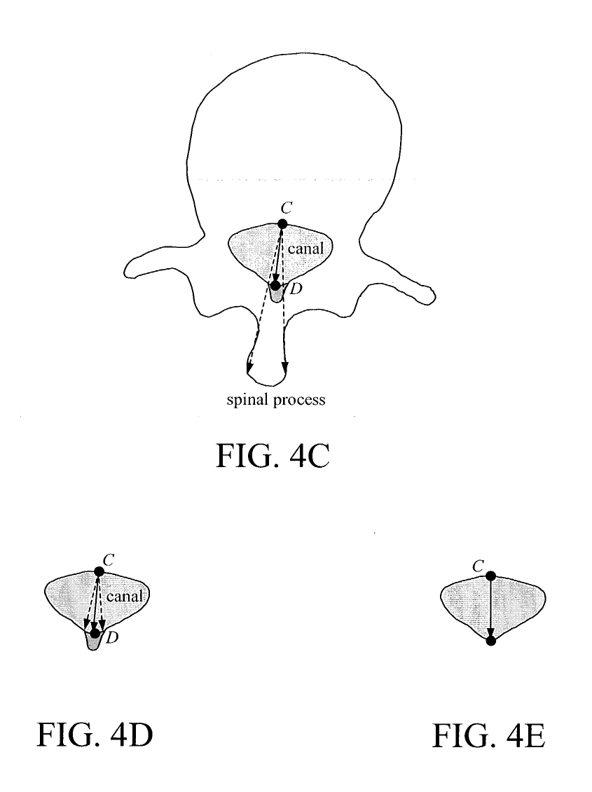

With reference to FIG. 2, computing the canal compression starts by obtaining the center of bone boundary displayed in each transverse sectional image. This is achieved by averaging the boundary points that represent the bone boundary (step 210), as shown by “center of bone tissue” in FIG. 4A. If the spine in the transverse sectional image contains other disc space, then the boundary of the disc space is also included to compute the center of bone tissue. The intersection points that the bone boundary makes with a 360-degree rotating vector extending from the center of bone tissue outward at individual integral angular positions constitute the vertebral boundary (step 220). If the vector makes more than one intersection with the bone boundary at each integral angular position, then it means that the vertebral body has a crack or hole. In this case, the outermost intersection point is the vertebral boundary. When the intersection points of two vectors have too large a distance, then the second vector is neglected. Examples of the neglected vector are those between rs(L) and rs(Ln) and between rs(R) and rs(Rn) in FIG. 4A. The B-spline curve is then used to approximate a possible boundary. Afterwards, the average value of each vertebral boundary point measured at each integral angular position (approximated by the B-spline curve) is taken to generate the vertebral center (step 230), as shown by “center of vertebral” in FIG. 4B. It should be noted that the above-mentioned cracks and holes are to be excluded when computing the vertebral center. The canal center is obtained from the left and right boundaries of the canal (step 240). That is, the bisecting vector of the vectors r(L) and r(R) in FIG. 4B is used to determine its intersection with the canal boundary. The intersection point, the point C in FIG. 4B, is the canal center. The method uses the vector extending through the canal to find the other boundary point corresponding to the canal center, point D in FIG. 4B, thereby determining the canal diameter. As the extracted transverse sectional image may or may not contain a spinal process or a transverse process, there are several different ways to compute the canal diameter. For example, the transverse sectional image in FIG. 4C has a spinal process, then the bisecting vector of the vectors on the left and right boundaries is used to find the canal center. The canal diameter is then determined from the canal center and its corresponding boundary point on the diagonal side. In FIG. 4D, there is no spinal or transverse process, then the bisecting vector of the vectors on the left and right boundaries of the disc space is used to find the location of the canal center. Similarly, the canal diameter is determined from the canal center and the corresponding boundary point on the diagonal side. In FIG. 4E, when no process or disc space exists in the transverse sectional image, a vector is directly pointing out from the canal center to find the corresponding diagonal boundary point for determining the canal diameter. After the canal diameter of each transverse sectional image is determined, the method then finds preliminary results for possible problematic spinal sectors from the variation of the canal diameter and, at the same time, determines the ratio and level to which the spine is compressed. The disclosed method takes the one that has the largest ratio and extent among all canal diameters to be the final result.

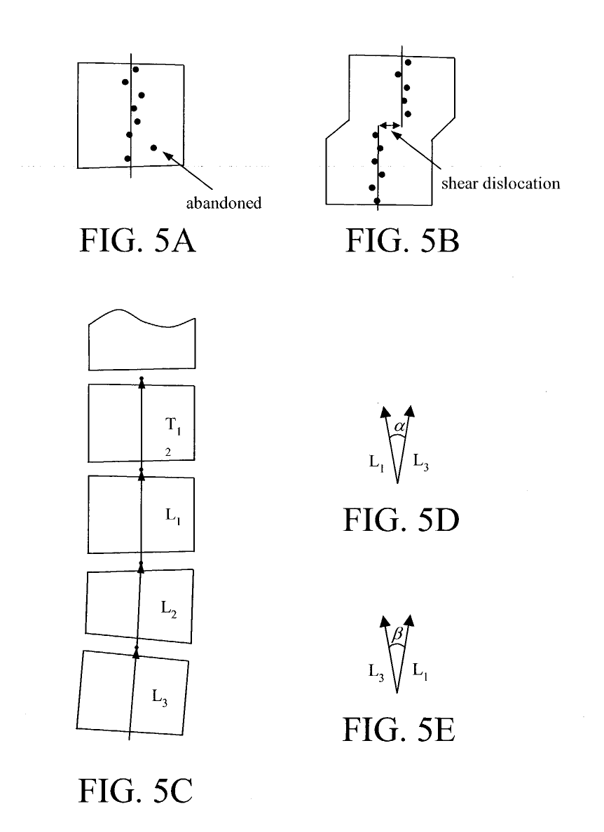

With reference to FIG. 3, the vertebral centers in the transverse sectional images are used to obtain the vertebral centerlines and their lengths (step 310). If some vertebral centers are too far away from the averaged vertebral center, then those points are abandoned and the vertebral centerline is recalculated, as shown in FIG. 5A. By comparing the distance between adjacent vertebral centerlines, one is able to learn the vertebral displacement (step 320). If there are more than one vertebral centerline with too large a shear dislocation in one vertebral body, then it means that the vertebral body has displacement occurred. In this case, the disclosed method considers each individual vertebral centerline as an independent analysis unit, as shown in FIG. 5B. The angles between adjacent vertebral centerlines are compared with normal angles to determine how curved the vertebral body is (step 330). Please refer to FIG. 5C, FIG. 5D, and FIG. 5E. If the angle is like in FIG. 5C, it means that the vertebral body is abnormally curved. The situation in FIG. 5D is normal. Finally, the vertebral body compression level is determined by comparing the lengths of adjacent vertebral centerlines (step 340). After the computation of spinal compression curvatures in all extracted transverse sectional images, the method can further provide accurate diagnostic analysis.

In the following, we use an actual case to illustrate that the disclosed method can indeed help diagnose the compression curvature on a spine.

A 39-year-old patient falls from a place six meters height from the ground. Clinical findings indicate that the patient has many symptoms of pain. From preliminary X-ray films, it is determined to take forty-eight transverse sectional images with the resolution of 256*256at an interval of 3 mm from the T10 sector to the L3 sector along the spine.

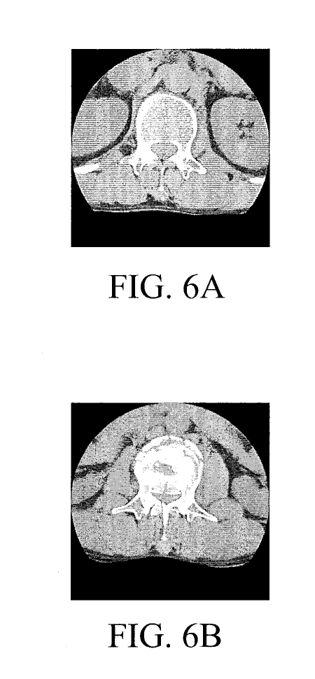

From the transverse sectional images in FIG. 6A and FIG. 6B, one can toughly see whether the spine is under pressure (FIG. 6A has a normal vertebral body, while FIG. 6B has a problematic one). However, visually reading the transverse sectional images cannot accurately gets hold of the actual situation about the compression curvature of the spine. Therefore, it is of not much use for medical diagnosis.

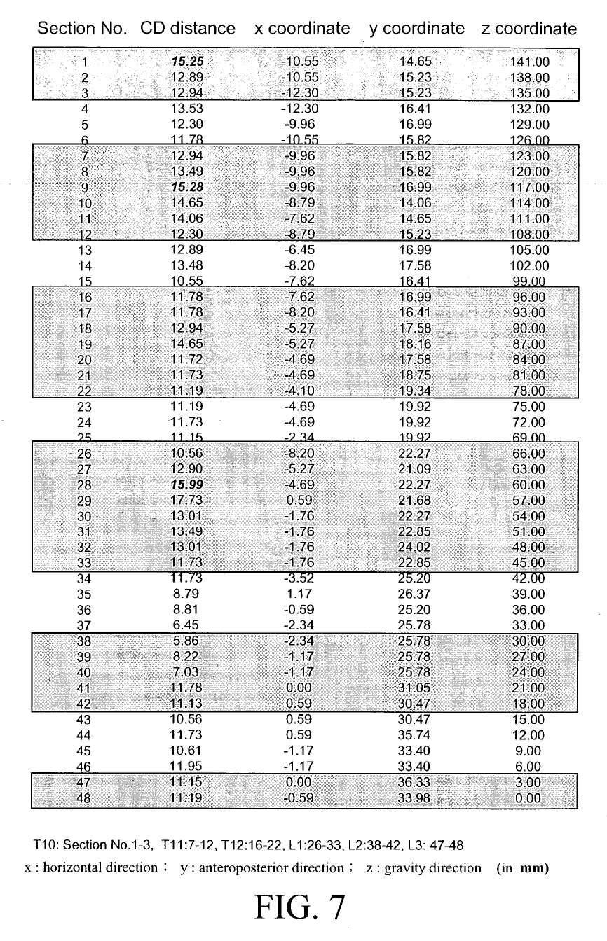

Through the analysis on the forty-eight transverse sectional images of various sectors along the patient spine, the disclosed method accurately determines such data as the canal diameter and the three-dimensional coordinates of the canal center in each transverse sectional image shown in FIG. 7. The canal diameters displayed in bold face are abandoned because of their large deviations from the average value. From FIG. 7 one sees that the canal diameters in the L2 sector are obviously shrunk; namely, the transverse sectional images No.38, No.39, and No.40. They show that the L2 sector is under pressure.

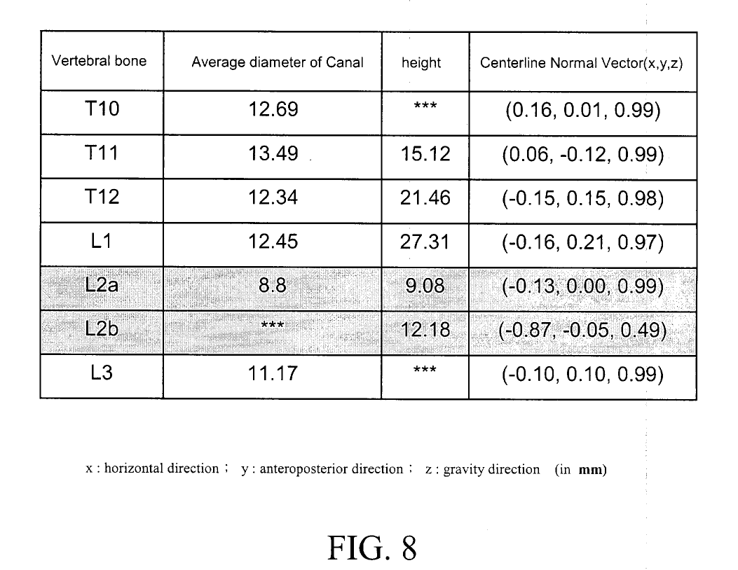

FIG. 8 further indicates that the diagnostic analysis covers the average canal diameter, the vertebral height, and the three-dimensional coordinates of the vertebral body. The L2 sector in the spine is considered as two independent analysis units (L2a and L2b) because the vertebral centerline is deviated too far. As the vertebral height of L2a is much shorter than the average vertebral height from other spinal sectors, this sector is therefore seriously depressed by external forces. Through the angle comparison from three-dimensional coordinates, one can know the angle, displacement, and height to be adjusted on the L2a sector.

After the disclosed method analyzes the transverse sectional images of the patient spine, one can understand the extent and type of the spine under pressure or with a fracture. Moreover, the method can provide data needed for surgical operations and therapeutic procedures. Therefore, the data computed by the invention can be displayed in terms of tables, transverse sectional image labels, or three-dimensional images according to different purposes.

Certain variations would be apparent to those skilled in the art, which variations are considered within the spirit and scope of the claimed invention.

What is claimed is:

1. An image analysis method for spinal compression curvature comprising the steps of:
extracting a plurality of transverse sectional images of the spine;
computing canal compression data for each of the extracted transverse sectional images;
finding a problematic spinal sector through the analysis of vertebral centerlines and computing curvature data for the problematic spinal sector; and
evaluating the extent and type of the problematic spine.

2. The method of claim 1, wherein the transverse sectional images are obtained from computed tomography (CT).

3. The method of claim 1, wherein the transverse sectional images are obtained from magnetic resonance imaging (MRI).

4. The method of claim 1, wherein the canal compression data include at least a canal diameter and three-dimensional coordinates of the canal center.

5. The method of claim 1, wherein the curvature data include at least an average canal diameter, a vertebral height, and three-dimensional coordinates of the vertebral centerline.

6. The method of claim 1, wherein the step of computing canal compression data for each of the extracted transverse sectional images further comprises the steps of:
using the averaged value of the boundary points of the bone in the transverse sectional image to obtain the center of bone tissue;
using a vector from the center of bone tissue toward each integral angular position to intersect with the outermost bone boundary, defining the intersection point as the vertebral boundary;
using the averaged value of the measured vertebral boundary points obtained at individual integral angular positions to determine a vertebral center;
using left and right boundary points of the canal to determine the canal center; and
extending the vector through the canal to find the canal diameter from the diagonal boundary point corresponding to the canal center.

7. The method of claim 1, wherein the step of finding a problematic spinal sector through the analysis of vertebral centerlines and computing curvature data for the problematic spinal sector further comprises the steps of:
using the vertebral centers in the transverse sectional images to obtain vertebral centerlines and vertebral centerline lengths;
comparing the dislocation between adjacent vertebral centerlines to obtain a vertebral displacement;
comparing the angle between adjacent vertebral centerlines with a normal angle to determine a curvature of the vertebral body; and
comparing the lengths of adjacent vertebral centerlines to obtain the compression level of the vertebral body.

8. The method of claim 1, wherein the data are displayed in terms of tables, transverse sectional image labels, and three-dimensional images.
